(19) United States
(12) Patent Application Publication
(10) Pub. No.: US 2022/0384014 A1
(43) Pub. Date: Dec. 1, 2022

(54) CARDIAC EPISODE CLASSIFICATION

(71) Applicant: Medtronic, Inc., Minneapolis, MN (US)

(72) Inventors: Shantanu Sarkar, Roseville, MN (US); Eduardo N. Warman, Maple Grove, MN (US); Ya-Jian Cheng, Lino Lakes, MN (US)

(21) Appl. No.: 17/329,913

(22) Filed: May 25, 2021

(51) Int. Cl. G16H 30/40; G16H 30/20; G16H 50/20; G16H 40/67; A61B 5/352

(52) U.S. Cl. CPC G16H 30/40; G16H 30/20; G16H 50/20; G16H 40/67; A61B 5/352; A61B 5/361

(57) ABSTRACT

A medical system includes communication circuitry configured to receive episode data for an episode sensed by a medical device of a patient, wherein the episode data comprises a cardiac electrogram sensed by the medical device during a period of time; and processing circuitry configured to generate an image based on the episode data, wherein the image is associated with an interval within the period of time; apply, by the processing circuitry, one or more machine learning models to the image, the one or more machine learning models configured to determine whether the image corresponds to an arrythmia type; and output an indication of whether the image corresponds to the arrythmia type.

### FIELD

This disclosure generally relates to medical devices and, more particularly, analysis of signals sensed by medical devices.

### BACKGROUND

Medical devices may be used to monitor physiological signals of a patient. For example, some medical devices are configured to sense cardiac electrogram (EGM) signals, e.g., electrocardiogram (ECG) signals, indicative of the electrical activity of the heart via electrodes. Some medical devices are configured to detect occurrences of cardiac arrhythmia, often referred to as episodes, based on the cardiac EGM and, in some cases, data from additional sensors. Example arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The medical devices may store the cardiac EGM and other data collected during a time period including an episode as episode data. The medical device may also store episode data for a time period in response to user input, e.g., from the patient.

A computing system may obtain episode data from medical devices to allow a clinician or other user to review the episode. A clinician may diagnose a medical condition of the patient based on identified occurrences of cardiac arrhythmias within the episode. In some examples, a clinician or other reviewer may review episode data to annotate the episodes, including determining whether arrhythmias detected by the medical device actually occurred, to prioritize the episodes and generate reports for further review by the clinician that prescribed the medical device for a patient or is otherwise responsible for the care of the particular patient.

### SUMMARY

In general, this disclosure describes techniques for classifying episode data, including cardiac EGM data, using one or more machine learning models. In some examples, processing circuitry applies one or more arrhythmia classification machine learning models to the episode data. The one or more machine learning models output, for example, a true or false indication for an arrhythmia type classification or a respective likelihood value representing the likelihood that a respective arrhythmia type classification occurred at any point during the episode.

According to one example, a computer-implemented method includes receiving, by processing circuitry of a medical device system, episode data for an episode stored by a medical device of a patient, wherein the episode data comprises a cardiac electrogram sensed by the medical device during a period of time; generating an image based on the episode data, wherein the image is associated with an interval within the period of time; applying, by the processing circuitry, one or more machine learning models to the image, the one or more machine learning models configured to determine whether the image corresponds to an arrythmia type; and outputting an indication of whether the image corresponds to the arrythmia type.

According to another example, a medical system includes communication circuitry configured to receive episode data for an episode sensed by a medical device of a patient, wherein the episode data comprises a cardiac electrogram sensed by the medical device during a period of time; processing circuitry configured to generate an image based on the episode data, wherein the image is associated with an interval within the period of time; apply, by the processing circuitry, one or more machine learning models to the image, the one or more machine learning models configured to determine whether the image corresponds to an arrythmia type; and output an indication of whether the image corresponds to the arrythmia type.

According to another example, a computer-readable storage medium stores instructions that when executed by one or more processors cause the one or more processors to receive episode data for an episode stored by a medical device of a patient, wherein the episode data comprises a cardiac electrogram sensed by the medical device during a period of time; generate an image based on the episode data, wherein the image is associated with an interval within the period of time; apply one or more machine learning models to the image, the one or more machine learning models configured to determine whether the image corresponds to an arrythmia type; and output an indication of whether the image corresponds to the arrythmia type.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of a medical device system configured to utilize machine learning models to detect cardiac arrhythmias in accordance with the techniques of the disclosure.

FIG. 2 is a block diagram illustrating an example configuration of the implantable medical device (IMD) of FIG. 1.

FIG. 3 is a conceptual side-view diagram illustrating an example configuration of the IMD of FIGS. 1 and 2.

FIG. 4 is a functional block diagram illustrating an example configuration of the computing system of FIG. 1.

FIGS. 5A and 5B is an example image template that may be used by the computing system of FIGS. 1 and 4 to generate an image when implementing the techniques of this disclosure.

FIGS. 5C-5H show examples of images generated based on the image template of FIGS. 5A and 5B.

FIGS. 6A-6D are process flow schematics illustrating an example operation for utilizing machine learning models to detect cardiac arrhythmias in accordance with the techniques of the disclosure.

FIG. 7 is a flow diagram illustrating an example operation for utilizing machine learning models to detect cardiac arrhythmias in accordance with the techniques of the disclosure.

Like reference characters refer to like elements throughout the figures and description.

### DETAILED DESCRIPTION

A variety of types of implantable and external medical devices detect arrhythmia episodes based on sensed cardiac EGMs and, in some cases, other physiological parameters. External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data, e.g., episode data for detected arrhythmia episodes, to a remote patient monitoring system, such as the Medtronic Carelink™ Network.

Implantable medical devices (IMDs) also sense and monitor cardiac EGMs, and detect arrhythmia episodes. Example IMDs that monitor cardiac EGMs include pacemakers and implantable cardioverter-defibrillators, which may be coupled to intravascular or extravascular leads, as well as pacemakers with housings configured for implantation within the heart, which may be leadless. Some IMDs that do not provide therapy, e.g., implantable patient monitors, sense cardiac EGMs. One example of such an IMD is the Reveal LINQ™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

By uploading episode data from medical devices, and distributing the episode data to various users, such network services may support centralized or clinic-based arrhythmia episode review. The episode data may include an indication of the one or more arrhythmias that the medical device detected during the episode. The episode data may also include data collected by the medical device during a time period including time before and after the instant the medical device determined the one or more arrhythmias to have occurred. The episode data may include the digitized cardiac EGM during that time period, heart rates or other parameters derived from the EGM during that time period, and any other physiological parameter data collected by the medical device during the time period.

This disclosure describes a system that uses machine learning to improve the ability of a medical device to detect and classify cardiac episodes. The medical device may, for example, be one of the devices described above or any other type of implantable device, such as a subcutaneous cardiac monitoring device, a single chamber ICD, an extravascular ICD, a subcutaneous ICD, or any other type of device configured to classify detected AF episodes. The system may also include an external device, such as a cloud-based system that is external to the cardiac monitoring device, like Medtronic Carelink™ Network introduced above.

Cardiac monitoring devices like those introduced above have the capability of detecting atrial fibrillation (AF) and other types of arrythmias. Despite various enhancements over the years to the AF detection algorithms, many cardiac monitoring devices still generate a significant number of inappropriate detections of AF episodes, which can cause a significant data review burden for physicians leading to a significant amount of time being spent on reviewing episodes rather than treating patients. The techniques of this disclosure may improve the accuracy of the AF detection and classification performed by the system, and/or the cardiac monitoring device specifically, and thus reduce the AF episode review time for physicians.

As cardiac monitoring devices are typically battery powered and, in the case of IMDs, need to have a sufficient enough battery life to justify implantation, the devices usually have limited processing capabilities in order to limit battery drain, which limits the complexity of the algorithms that can be implemented inside the cardiac monitoring device. Thus, cardiac monitoring devices can be configured to transmit data collected for suspected cardiac episodes to the external system so the external system can use advanced signal processing techniques to post process stored and transmitted data for episodes prior to review by physicians. This disclosure describes advanced signal processing techniques that may be used by the external system to post process the AF episodes detected by the cardiac monitoring device. Although the techniques of this disclosure will be described as being performed by an external system, it should be understood that in other implementations the described techniques may be performed by the IMD itself.

One existing signal processing technique includes feeding ECG data into a neural network to train the neural network to recognize AF. This disclosure describes techniques for generating transformed data from raw ECG data in the form of 1D vectors or 2D vectors (e.g., images) of transformed data. The transformed data may include additional data beyond just unprocessed ECG data, to train a neural network to classify a cardiac episode, such as an AF episode, as being either true or false, with true meaning an actual AF event occurred and false meaning the detected event was not AF. In some examples, using the neural network, the system may provide a confidence score, such as 0-100% or 1-10, indicating a likelihood that the AF episode is true. In yet other examples, the neural network may additionally or alternatively determine a classification for a cardiac episode. Examples of classifications include AF, right atrial flutter, left atrial flutter, runs of PACs, short bursts of AT/AF, N:1 AT, 1:1 AT, sinus with ectopy, bigeminy, trigeminy, runs of PVCs, myopotential noise, EMI, T-wave oversensing, P-wave oversensing, sinus tachycardia, and so on. The different images may, for example, include features extracted from the raw ECG data, rather than the raw ECG data itself. In this context, raw ECG data generally refers to the digitized cardiac EGM.

According to a first technique of this disclosure, the external system may process stored episode data to extract RR intervals, convert the RR intervals into a Lorenz plot of delta RR intervals, and then classify the Lorenz plot using a deep learning network. Specifically, the deep learning network may classify an image of the Lorenz plot. According to one example implementation, the external system may use a 2D convolution neural network to extract features from the Lorenz plot and train a neural network which can then be used to classify newly detected AF episodes.

Additionally or alternatively, according to a second technique of this disclosure, the external system may process stored episodes to determine ECG segments prior to R-wave senses, and then convert the ECG segments into a 2D overlay in which each row corresponds to each R-wave sense and each column corresponds to the ECG signal amplitude prior to R-wave sense. The external system may process the ECG using a log transformation to normalize the R-wave amplitudes and enhance the P-waves and baseline prior to the R-wave. Then, the external system may use an R-wave sense synced 2D ECG overlay to classify new episode data using a deep learning network. According to one example implementation, the external system may use a 2D convolution neural network to extract features from the 2D ECG overlay and train a neural network which can then be used to classify newly detected arrythmias, such as AF episodes.

Additionally or alternatively, according to a third technique of this disclosure, the external system may use a neural network or other machine learning techniques like the examples described above in conjunction with other features such as episode duration, premature atrial contraction (PAC) count, premature ventricular contractions (PVC) count, entropy, AF evidence score, P-wave evidence score, etc. to classify newly detected AF episodes. That is, the external system may implement multiple, independent, classification techniques or algorithms to classify an episode as either corresponding to an arrythmia type or not. The system may then make a final determination based on the multiple, independent, classification techniques or algorithms via an averaging or voting mechanism, for example.

Using any combination or permutation of the techniques described above, the external system may generate an image. As will be described in more detail, the generated image may include multiple sub-images, with each sub-image representing a different feature or characteristic of the episode data. These features or characteristics may, for example, be any of a Lorenz plot determined from the ECG data, a normalized Lorenz plot determined from the ECG data, a Lorenz plot of RR intervals or deltaRR intervals or a combination of RR and deltaRR intervals, a multi-scale RR histogram determined from the ECG data, or an episode duration encoding block determined from the ECG data. The normalized Lorenz plot may, for example, be normalized to a scale of 0-255 or to some other such scale. Another example of a feature that may be used is a Lorenz plot of delta RR interval that is normalized to the median RR interval (e.g., over an entire 2 minute episode or the median for the 4 beats used for current and previous deltaRR interval).

Other examples of features may include a spectrogram of the ECG data, a wavelet transformation of the ECG data, a principal component analysis (PCA) of the ECG data. Other data, such as a recent AF burden history for a patient and probability scores of recently classified episodes, may also be used by the external system to classify an arrythmia.

In this disclosure, an image that includes multiple sub-images may be referred to herein as a combined image. In one example implementation of the techniques of this disclosure, the external system may apply one or more machine learning models to a combined image to classify an episode. In another implementation of the techniques of this disclosure, the external system may apply one or more machine learning models individually to one or more sub-images to classify the episode. The external system may use multiple fully connected networks to create an ensemble network. That is, the external system may use a plurality of different machine learning models, applied to a combined image and/or sub-images, and based on the determinations of the multiple different machine learning modes, determine a final classification for an episode.

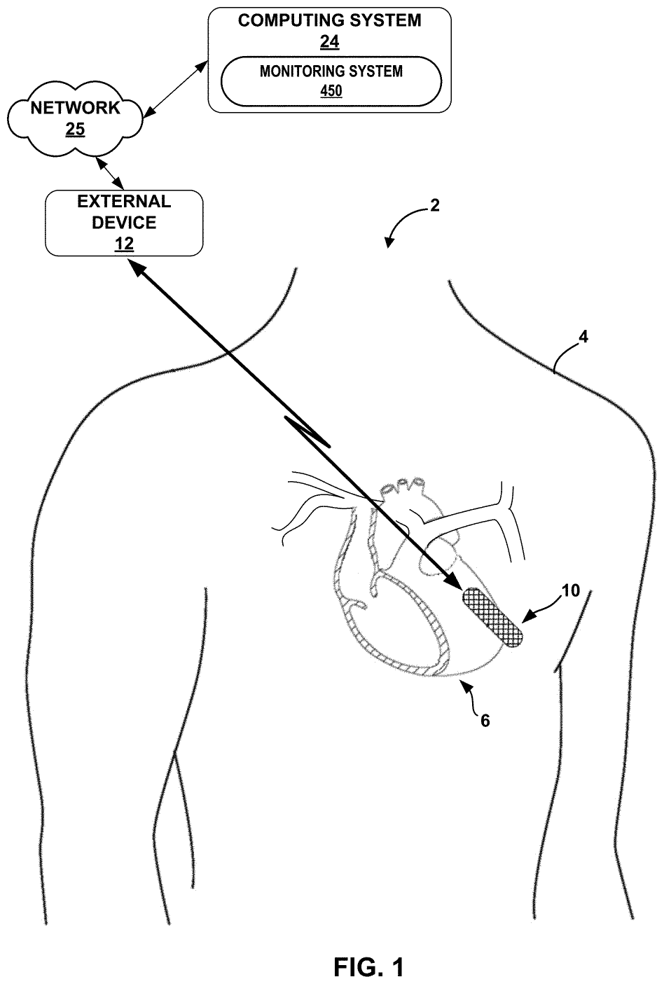

FIG. 1 is a conceptual drawing illustrating an example of a medical device system 2 configured to utilize machine learning models to detect cardiac arrhythmias in accordance with the techniques of the disclosure. The example techniques may be used with an IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM. Although described primarily in the context of examples in which the medical device that collects episode data takes the form of an ICM, the techniques of this disclosure may be implemented in systems including any one or more implantable or external medical devices, including monitors, pacemakers, or defibrillators.

External device 12 is a computing device configured for wireless communication with IMD 10. External device 12 may be configured to communicate with computing system 24 via network 25. In some examples, external device 12 may provide a user interface and allow a user to interact with IMD 10. Computing system 24 may comprise computing devices configured to allow a user to interact with IMD 10, or data collected from IMD, via network 25.

External device 12 may be used to retrieve data from IMD 10 and may transmit the data to computing system 24 via network 25. The retrieved data may include values of physiological parameters measured by IMD 10, indications of episodes of arrhythmia or other maladies detected by IMD 10, episode data collected for episodes, and other physiological signals recorded by IMD 10. The episode data may include EGM segments recorded by IMD 10, e.g., due to IMD 10 determining that an episode of arrhythmia or another malady occurred during the segment, or in response to a request to record the segment from patient 4 or another user.

In some examples, computing system 24 includes one or more handheld computing devices, computer workstations, servers or other networked computing devices. In some examples, computing system 24 may include one or more devices, including processing circuitry and storage devices, that implement a monitoring system 450. Computing system 24, network 25, and monitoring system 450 may be implemented by the Medtronic Carelink™ Network or other patient monitoring system, in some examples.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

Monitoring system 450, e.g., implemented by processing circuitry of computing system 24, may implement the techniques of this disclosure including applying machine learning models to episode data to detect cardiac arrhythmias. Monitoring system 450 may receive episode data for episodes from medical devices, including IMD 10, which may store the episode data in response to their detection of an arrhythmia and/or user input. Based on the application of one or more arrhythmia classification machine learning models, monitoring system 450 may determine the likelihood that one or more arrhythmias of one or more types occurred during the episode including, in some examples, the arrhythmia identified by the medical device that stored the episode data.

Monitoring system 450 may, for example, receive episode data, e.g., ECG data, for an episode of a patient from IMD 10 and generate an image based on the episode data. Monitoring system 450 may then apply one or more machine learning models to the image to determine whether the image corresponds to an arrythmia type, such as AF. As will be explained in more detail below, the image may include a single image for a single feature or a plurality of sub-images, with each sub-image corresponding to one or more features extracted or derived from the ECG data. These features or characteristics may, for example, be any of a Lorenz plot determined from the ECG data, a normalized Lorenz plot determined from the ECG data, a multi-scale RR histogram determined from the ECG data, or an episode duration encoding block determined from the ECG data.

In some examples, monitoring system 450 may process stored episode data from IMD 10 to extract RR intervals, convert the RR intervals into a Lorenz plot of delta RR intervals, and then classify an image of the Lorenz plot using a deep learning network. Monitoring system 450 may, for example, use a 2D convolution neural network to extract features from the Lorenz plot and train a neural network which can then be used to classify newly detected arrythmias, such as AF episodes. Additionally or alternatively, monitoring system 450 may process stored episode data from IMD 10 to determine ECG segments prior to R-wave senses, and then convert the ECG segments into a 2D overlay with each row corresponding to each R-wave sense and each column corresponding to the ECG signal amplitude prior to R-wave sense. Monitoring system 450 may process the ECG using a log transformation to normalize the R-wave amplitudes and enhance the P-waves and baseline prior to the R-wave, and then use an R-wave sense synced 2D ECG overlay to classify new episode data using a deep learning network. According to one example implementation, the external system may use a 2D convolution neural network to extract features from the 2D ECG overlay and train a neural network which can then be used to classify newly detected AF episodes.

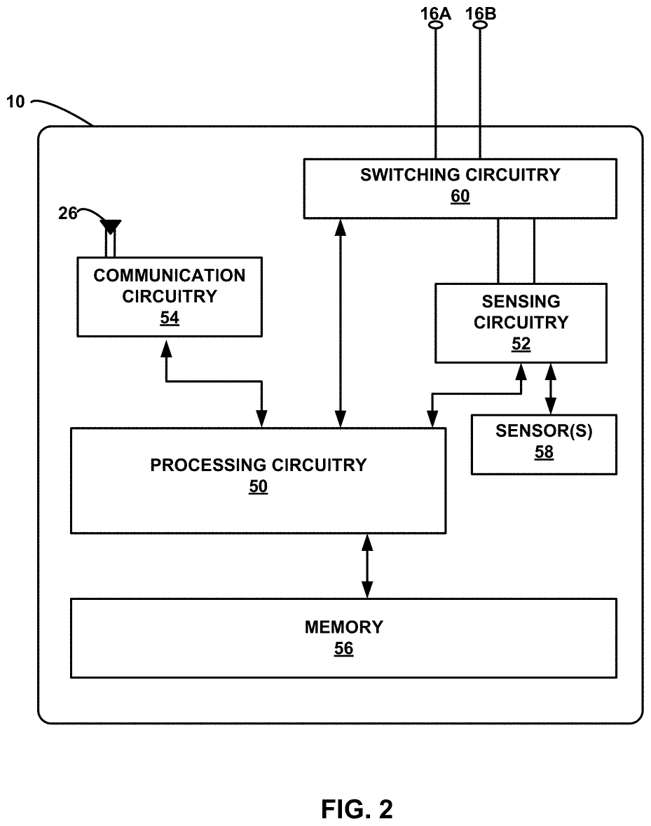

FIG. 2 is a block diagram illustrating an example configuration of IMD 10 of FIG. 1. As shown in FIG. 2, IMD 10 includes processing circuitry 50 sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor electrical activity of a heart of patient 4 of FIG. 1 and produce cardiac EGM data for patient 4. In some examples, processing circuitry 50 may identify features of the sensed cardiac EGM to detect an episode of cardiac arrhythmia of patient 4. Processing circuitry 50 may store the digitized cardiac EGM and features of the EGM used to detect the arrhythmia episode in memory 56 as episode data for the detected arrhythmia episode. In some examples, processing circuitry 50 stores one or more segments of the cardiac EGM data, features derived from the cardiac EGM data, and other episode data in response to instructions from external device 12 (e.g., when patient 4 experiences one or more symptoms of arrhythmia and inputs a command to external device 12 instructing IMD 10 to upload the data for analysis by a monitoring center or clinician).

In some examples, processing circuitry 50 transmits, via communication circuitry 54, the episode data for patient 4 to an external device, such as external device 12 of FIG. 1. For example, IMD 10 sends digitized cardiac EGM and other episode data to network 25 for processing by monitoring system 450 of FIG. 1.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining features of the cardiac EGM including inter-depolarization intervals, heart rate, and detecting arrhythmias, such as tachyarrhythmias and asystole. Sensing circuitry 52 may also provide one or more digitized cardiac EGM signals to processing circuitry 50 for analysis, e.g., for use in cardiac rhythm discrimination and/or to identify and delineate features of the cardiac EGM, such as QRS amplitudes and/or width, or other morphological features.

In some examples, IMD 10 includes one or more sensors 58, such as one or more accelerometers, microphones, optical sensors, and/or pressure sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to identify arrhythmia episodes and stored as episode data in memory 56.

Communication circuitry 54 may include any suitable hardware, firmware, software or any combination thereof for communicating with another device, such as external device 12. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device via an external device (e.g., external device 12) and a computer network, such as the Medtronic CareLink® Network developed by Medtronic, plc, of Dublin, Ireland.

Although described herein in the context of example IMD 10, the techniques for cardiac arrhythmia detection disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device external to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, or a “smart” watch.

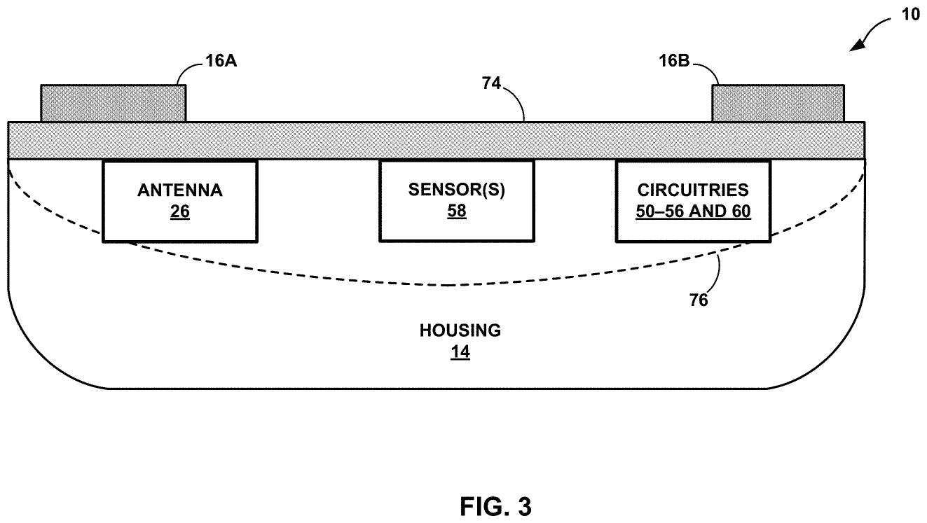

FIG. 3 is a conceptual side-view diagram illustrating an example configuration of IMD 10. In the example shown in FIG. 3, IMD 10 may include a leadless, subcutaneously-implantable monitoring device having a housing 14 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 14. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 14 such that housing 14 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 14. When flipped and placed onto housing 14, the components of IMD 10 formed on the inner side of insulative cover 74 may be positioned in a gap 76 defined by housing 14. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

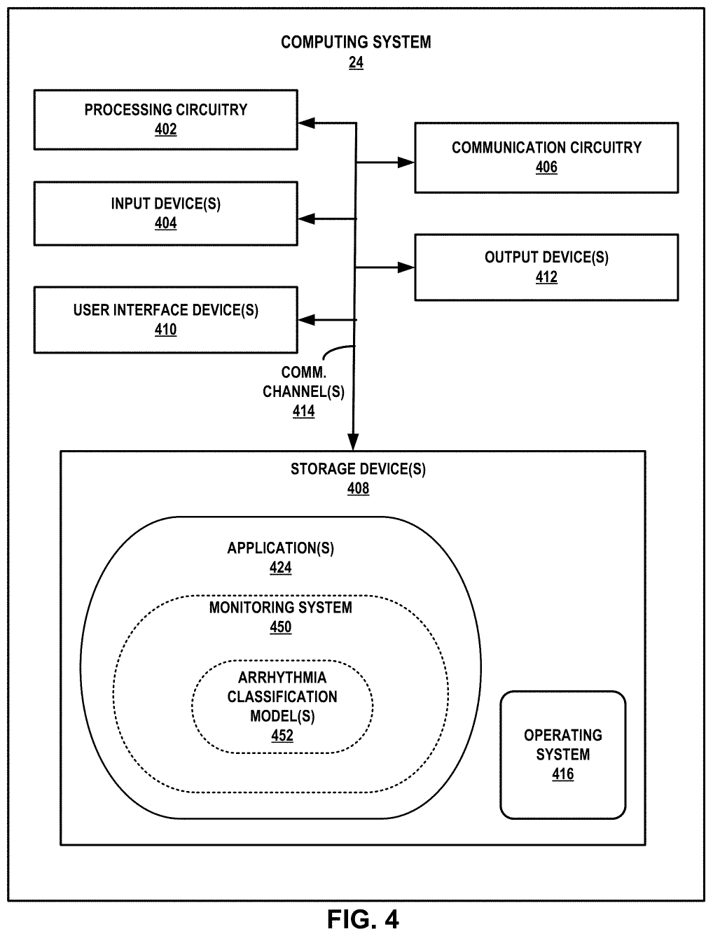

FIG. 4 is a block diagram illustrating an example configuration of computing system 24. In the illustrated example, computing system 24 includes processing circuitry 402 for executing applications 424 that include monitoring system 450 or any other applications described herein. Computing system 24 may be any component or system that includes processing circuitry or other suitable computing environment for executing software instructions and, for example, need not necessarily include one or more elements shown in FIG. 4 (e.g., input devices 404, communication circuitry 406, user interface devices 410, or output devices 412; and in some examples components such as storage device(s) 408 may not be co-located or in the same chassis as other components). In some examples, computing system 24 may be a cloud computing system distributed across a plurality of devices.

In the example of FIG. 4, computing system 24 includes processing circuitry 402, one or more input devices 404, communication circuitry 406, one or more storage devices 408, user interface (UI) device(s) 410, and one or more output devices 412. Computing system 24, in some examples, further includes one or more application(s) 424 such as monitoring system 450, and operating system 416 that are executable by computing system 24. Each of components 402, 404, 406, 408, 410, and 412 may be coupled (physically, communicatively, and/or operatively) for inter-component communications. In some examples, communication channels 414 may include a system bus, a network connection, an inter-process communication data structure, or any other method for communicating data. As one example, components 402, 404, 406, 408, 410, and 412 may be coupled by one or more communication channels 414.

Processing circuitry 402, in one example, is configured to implement functionality and/or process instructions for execution within computing system 24. For example, processing circuitry 402 may be capable of processing instructions stored in storage device 408. Examples of processing circuitry 402 may include any one or more of a microprocessor, a controller, a digital signal processor (DSP), an application specific integrated circuit (ASIC), a field-programmable gate array (FPGA), or equivalent discrete or integrated logic circuitry.

One or more storage devices 408 may be configured to store information within computing device 400 during operation. Storage device 408, in some examples, is described as a computer-readable storage medium. In some examples, storage device 408 is a temporary memory, meaning that a primary purpose of storage device 408 is not long-term storage. Storage device 408, in some examples, is described as a volatile memory, meaning that storage device 408 does not maintain stored contents when the computer is turned off. Examples of volatile memories include RAM, dynamic random access memories (DRAM), static random access memories (SRAM), and other forms of volatile memories known in the art. In some examples, storage device 408 is used to store program instructions for execution by processing circuitry 402. Storage device 408, in one example, is used by software or applications 424 running on computing system 24 to temporarily store information during program execution.

Storage devices 408, in some examples, also include one or more computer-readable storage media. Storage devices 408 may be configured to store larger amounts of information than volatile memory. Storage devices 408 may further be configured for long-term storage of information. In some examples, storage devices 408 include non-volatile storage elements. Examples of such non-volatile storage elements include magnetic hard discs, optical discs, floppy discs, flash memories, or forms of electrically programmable memories (EPROM) or electrically erasable and programmable memories (EEPROM).

Computing system 24, in some examples, also includes communication circuitry 406 to communicate with other devices and systems, such as IMD 10 and external device 12 of FIG. 1. Communication circuitry 406 may include a network interface card, such as an Ethernet card, an optical transceiver, a radio frequency transceiver, or any other type of device that can send and receive information. Other examples of such network interfaces may include 3G and WiFi radios.

Computing system 24, in one example, also includes one or more user interface devices 410. User interface devices 410, in some examples, are configured to receive input from a user through tactile, audio, or video feedback. Examples of user interface devices(s) 410 include a presence-sensitive display, a mouse, a keyboard, a voice responsive system, video camera, microphone or any other type of device for detecting a command from a user. In some examples, a presence-sensitive display includes a touch-sensitive screen.

One or more output devices 412 may also be included in computing system 24. Output devices 412, in some examples, are configured to provide output to a user using tactile, audio, or video stimuli. Output devices 412, in one example, include a presence-sensitive display, a sound card, a video graphics adapter card, or any other type of device for converting a signal into an appropriate form understandable to humans or machines. Additional examples of output devices 412 include a speaker, a cathode ray tube (CRT) monitor, a liquid crystal display (LCD), or any other type of device that can generate intelligible output to a user.

Computing system 24 may include operating system 416. Operating system 416, in some examples, controls the operation of components of computing system 24. For example, operating system 416, in one example, facilitates the communication of one or more applications 424 and monitoring system 450 with processing circuitry 402, communication circuitry 406, storage device 408, input device 404, user interface devices 410, and output device 412.

Applications 424 may also include program instructions and/or data that are executable by computing device 400. Example application(s) 424 executable by computing device 400 may include monitoring system 450. Other additional applications not shown may alternatively or additionally be included to provide other functionality described herein and are not depicted for the sake of simplicity.

In accordance with the techniques of the disclosure, computing system 24 receives episode data for episodes stored by medical devices, such as IMD 10, via communication circuitry 406. Storage device 408 may store the episode data for the episodes in storage device 408. The episode data may have been collected by the medical devices in response to the medical devices detecting arrhythmias and/or user input directing the storage of episode data.

Monitoring system 450, as implemented by processing circuitry 402, may review and annotate the episodes, and generate of reports or other presentations of the episodes subsequent to the annotation for review by a clinician or other reviewer. Monitoring system 450 may utilize input devices 404, output devices 412, and/or communication circuitry 406 to display episode data, arrhythmia type classifications, and any other information described herein to users, and to receive any annotations or other input regarding the episode data from the users.

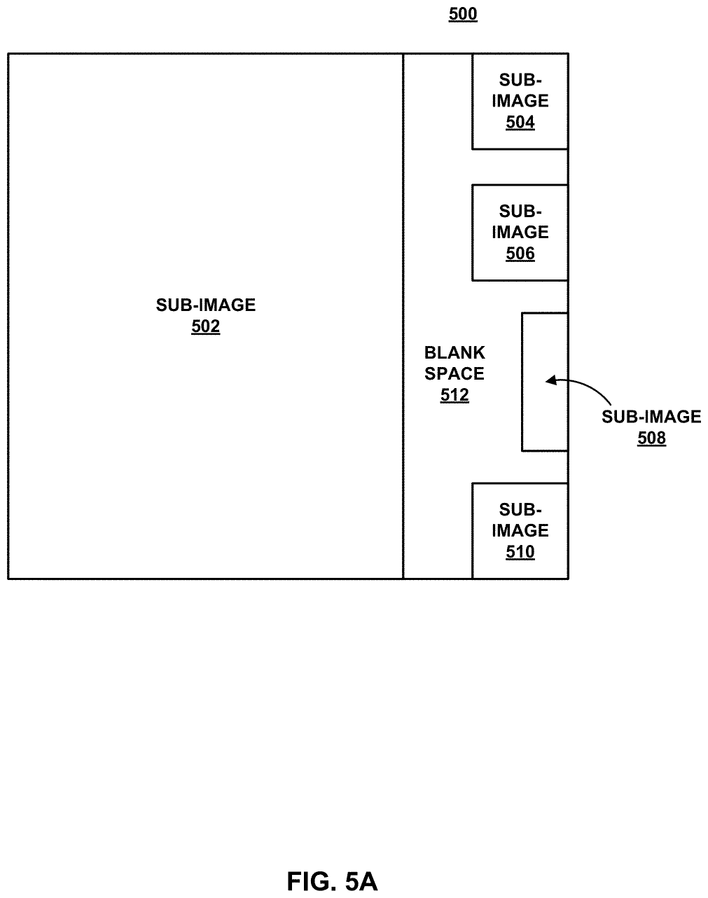

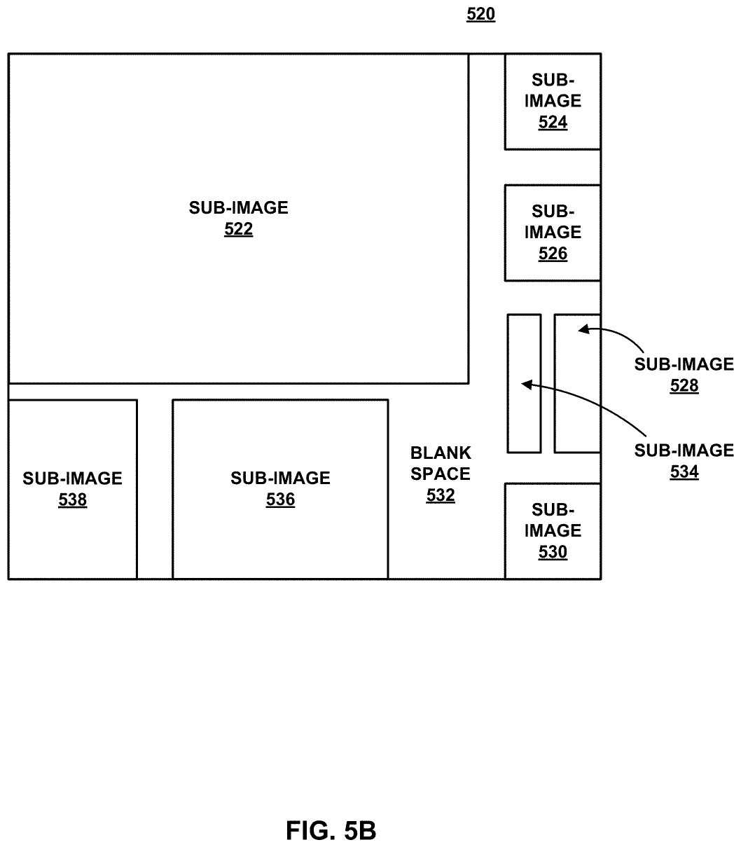

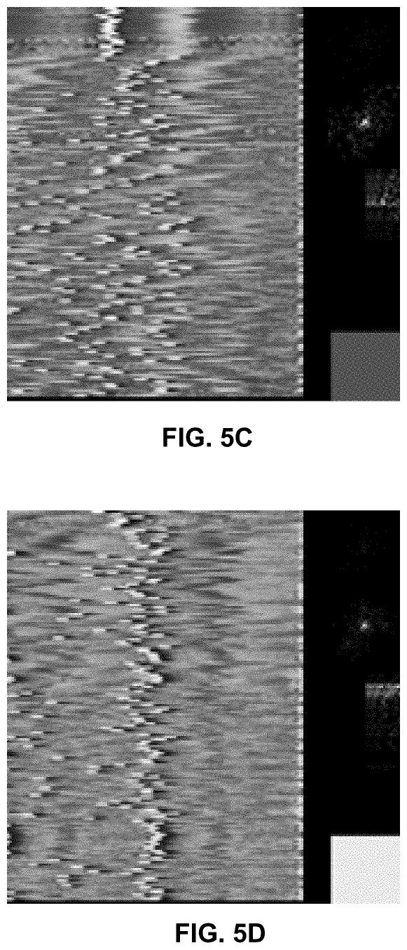

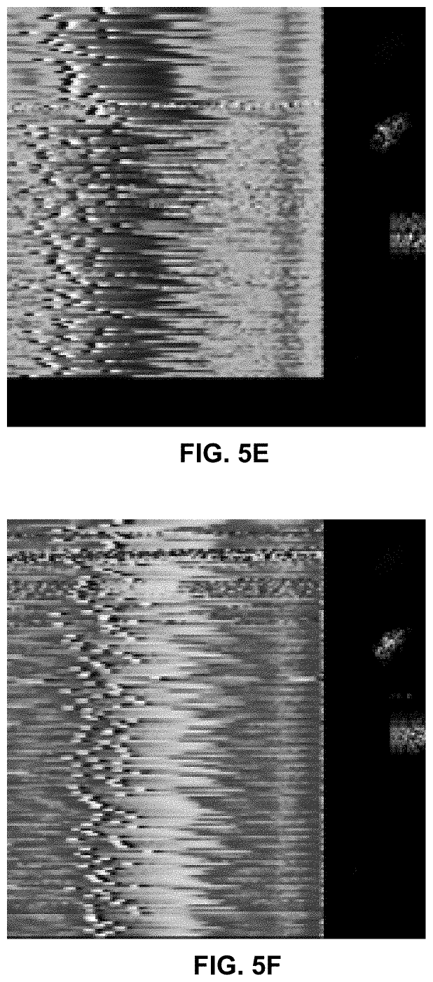

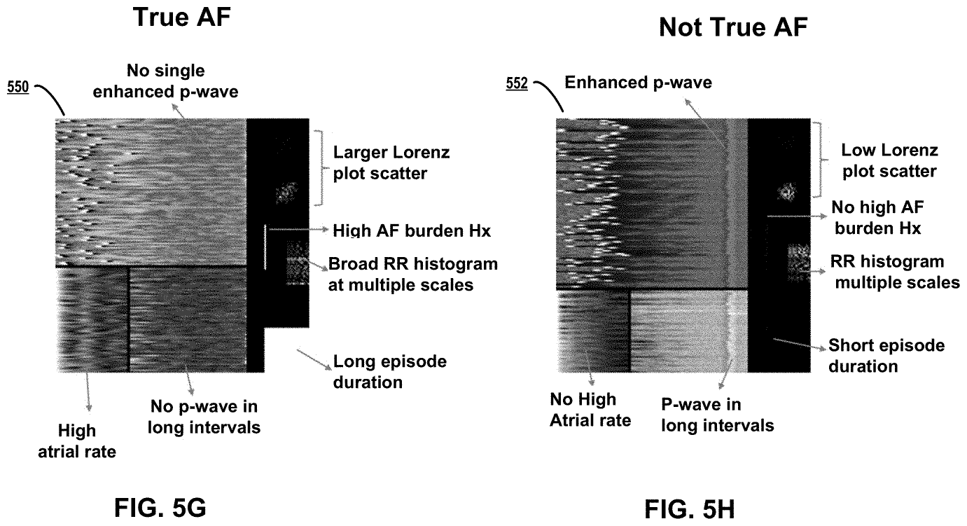

Monitoring system 450 may receive, from IMD 10, episode data for an episode and generate an image, or more generally a 2D array of numbers, based on the episode data. FIG. 5A shows an example of image template 500, which represents an example layout for an image that may be generated by monitoring system 450. Image template 500 may have any resolution or bit depth. In some examples, image template 500 may have a bit depth of one, meaning that each pixel in image template 500 has a value of either 0 or 1, which conceptually may correspond to either black or white. In other examples, image template 500 may have a bit depth n, with n being than one, meaning that each pixel in image template 500 can have a value ranging from 0 to 2ⁿ−1, which conceptually may correspond to either black, white, or various shades of gray.

Image template 500 includes sub-image 502, sub-image 504, sub-image 506, sub-image 508, sub-image 510, and blank space 512. Sub-image 502, sub-image 504, sub-image 506, sub-image 508, sub-image 510 may each include image data generated based on features of the episode data. Blank space 512 may represent a border or buffer between sub-images that does not include any sort of image data for features of the episode data. That is, all values within blank space 512 may be set to the same pixel value.

In one example implementation, image template 500 may have a resolution of 170×170, and sub-image 502 may have a resolution of 170×128. Sub-image 504, sub-image 506, and sub-image 510 may each have a resolution of 31×31, and sub-image 508 may have a resolution 41×15. Monitoring system 450 may, for example, include an image of normalized R-wave amplitudes in sub-image 502, with each of the 170 rows corresponding to an interval for the R-wave (e.g., 128 samples prior to the R-wave). If more than 170 intervals are present, then sub-image 502 may, for example, include the first 85 intervals and last 85 intervals, or some other such sub-sampling of the intervals. Monitoring system 450 may, for example, also include an image of a Lorenz plot in sub-image 504, an image of a normalized Lorenz plot in sub-image-506, an image of a multi-scale RR histogram in sub-image 508, and an image of an episode duration encoding block in sub-image 510.

Monitoring system 450 may, for example, generate the image for sub-image 502 by transforming the ECG data. An example of a transform process performed by monitoring system 450 may include:
(1) Subtract median value of ECG samples (over 2 minutes) from each ECG sample.
(2) Compute the 1st percentile and 99th percentile of the ECG amplitudes after step 1.
(3) If abs(1st p-tile)>abs(99th p-tile) then it means R-wave has negative polarity, then flip the ECG to have all ECGs with +ve polarity.
(4) Take natural log(ECG signal*0.1+1). Do positive signal and negative signal separately and combine them to preserve polarity.

(5) Normalize transformed ECG in (4) from 0-255 for gray scale values of image.

Monitoring system 450 may then use the normalized ECG values to populate sub-image 502, such that sub-image 502 includes each R-wave sense for each row and then the log transformed ECG data samples (e.g., 128 samples) look back from the R-wave sense.

Monitoring system 450 may, for example, include an RR interval histogram in sub-image 508, which in this example is a 41×15 image. Each of the rows may, for example, include a histogram of RR intervals split into 41 bins, spanning from 400 ms to 1200 ms with a bin size of 20 ms. Intervals less than 400 may be set to 400, and intervals greater than 1200 may be set to 1200. The first column is the histogram of RR intervals for the entire 2 minutes, and the next two columns are the histogram of RR intervals for the first half and last half of the total number of beats. The next four columns split the number of beats into 4 segments before doing the histogram of RR intervals, and then the last 8 columns are histogram of RR intervals which splits number of beats into 8 segments. The splits may be based on a number of beats or an amount of time. Monitoring system 450 may normalize the histograms, e.g., 8-splits may be multiplied by 8, 4-splits multiplied by 4, 2 splits multiplied by 2. This may help extract from the data the variability of RR intervals as well as whether any rate onset or offset is present.

Monitoring system 450 may, for example, encode an episode duration into a 2D image within sub image 510, by having each pixel in sub image 510 be progressively populated depending on episode duration. The indexing may, for example, increment every 2 minutes up to 1 hour, such that for a 2 minute episode, monitoring system 450 only fills one pixel in a 30×30 image, populates a 5×5 block for 10 minutes, or the entire 30×30 block for durations of one hour or longer. The intensity of the pixel may be determined by the actual episode duration in minutes, going from 2 to 255 minutes, for example.

Image template 500 merely represents one example of an image that monitoring system 450 may generate when performing the techniques of this disclosure. The particular arrangement of features with respect to sub-images 502, 504, 506, 508, and 510 represents only one example of the many arrangements contemplated by this disclosure. In other examples, monitoring system 450 may be configured to generate images of other shapes and sizes (e.g., resolutions), and/or sub-images of different shapes and sizes. Moreover, monitoring system 450 may be configured to generate images with more or fewer sub-images, and the sub-images may include image data representing different features or a different arrangement of features than those described above. The sizes of sub-images may be selected based on the significance or weight to be applied to a certain feature. Generally speaking, when compared to a sub-image with fewer pixels, a sub-image with more pixels more strongly influences the machine learning models' determinations of whether an image corresponds to true AF or not. First, a larger number of pixels representing a particular feature leads to more feature weights that the model can fit to. Secondly, it also lets those features contribute significantly in the dense layer after multiple max pooling operations. For example, the duration of an AF episode theoretically could be conveyed using a single pixel, with the values of 0-255 corresponding to different amounts of time. By using a 30×30 size block, however, in the manner described above with respect to sub-image 510, monitoring system 450 applies more weight to the feature of episode duration in determining a classification for an arrythmia.

FIG. 5B shows an example of image template 520, which represents an example layout for an image that may be generated by monitoring system 450. Image template 520 may have any resolution or bit depth. In some examples, image template 520 may have a bit depth of one, meaning that each pixel in image template 520 has a value of either 0 or 1, which conceptually may correspond to either black or white. In other examples, image template 520 may have a bit depth n, with n being than one, meaning that each pixel in image 520 can have a value ranging from 0 to 2ⁿ−1 (e.g. 0-255), which conceptually may correspond to either black, white, or various shades of gray.

Image template 520 includes sub-image 522, sub-image 524, sub-image 526, sub-image 528, sub-image 530, and blank space 532. Sub-image 522, sub-image 524, sub-image 526, sub-image 528, sub-image 530 may each include image data generated based on features of the episode data, as described above for sub-image 502, sub-image 504, sub-image 506, sub-image 508, and sub-image 510, respectively. Blank space 532 may represent a border or buffer between sub-images that does not include any sort of image data for features of the episode data. That is, all values within blank space 512 may be set to the same pixel value. Blank space 532 may represent a border or buffer between sub-images that does not include any sort of image data for features of the episode data. That is, all values within blank space 512 may be set to the same pixel value.

Image template 520 also includes sub-image 534, sub-image 536, and sub-image 538. Sub-image 534 may, for example, include image date indicative of an AF burden, e.g., how many AF episodes a patient has had over a certain time period, such as the last 30 days. In one example, the number of hours per day over the last 30 days which a patient has experienced AF may be scaled by 10 and mapped to a range of 0-255.

Sub-image 536 may, for example, include image data indicative of P-waves, and sub-image 538 may include image data indicative of an estimated atrial rate. In cases where there are long intervals, such as an RR greater than 780 ms (or some other threshold) and the absolute difference between a current RR and a previous RR or the absolute difference between a current RR and a next RR is greater than 100 ms (or some other threshold), then monitoring system 450 may be configured to extract and store a segment (e.g., 600 ms) of the ECG waveform prior to the R-wave. If the number of such intervals is greater than a value, such as 5, then monitoring system 450 may create an additional sub-image, such as sub-image 536, for P-waves for long intervals.

Monitoring system 450 may determine an ensemble average for these extracted long interval P-wave signals and the values to sub-image 536. Monitoring system 450 may then cross-correlate each signal (or row of sub-image of 536) with the ensemble average and shift by a lag where cross correlation is highest, such that P-wave signals are maximally aligned. Monitoring system 450 may then ensemble average the shifted signals to create an additional average signal of maximally aligned signal and add this average signal as a row sub-image 536.

Monitoring system 450 may perform an autocorrelation of each row of this image (with aligned P-wave segments, and the two ensemble averages-aligned and not aligned) to form the atrial rate estimation image, e.g., sub-image 538. If there are multiple P-waves between two R-waves, for example in atrial tachycardia or atrial flutter or in some forms of atrial fibrillation, then the autocorrelation may show a second peak at the lag that corresponds to the cycle length. The P-wave image (e.g., sub-image 536) may, for example, be Ax78 pixels where A is determined by a number of long intervals with variability as described above. A may also be dependent on the number of rows used for sub-image 522. The autocorrelation image (e.g., sub-image 538) may be Ax48 pixels.

FIGS. 5C and 5D show examples of images generated based on the image template of FIG. 5A. FIGS. 5C and 5D are examples of true AF. The sub-image in the regions of FIGS. 5C and 5C that correspond to sub-image 502 have an absence of a single P-wave between two R-waves, which is indicative of AF. The sub-images in the regions of FIGS. 5C and 5D that corresponds to sub-images 504 and 506 of FIG. 5A show Lorenz plot sub-images and normalized Lorenz plot sub images with more scatter, which is also indicative of AF. The sub-images in the regions of FIGS. 5C and 5D that corresponds to sub-image 510 of FIG. 5A show a longer episode duration, which is indicative of AF. The sub-image in the region of FIG. 5B that corresponds to sub-images 508 shows a histogram with a bright spot in a subscale image, which may indicate rate onset, otherwise a variable RR.

FIGS. 5E and 5F show examples of images generated based on the image template of FIG. 5B. FIGS. 5E and 5F show examples normal (i.e., non-AF) sinus rhythms with variable RR intervals. The regions of FIGS. 5E and 5F that correspond to sub-mage 502 of FIG. 5A show evidence of single P-waves between two R-waves, which can be seen from the alternate white and dark shading along the vertical towards the right side of the regions corresponding to sub-image 502. The other regions corresponding to sub-images 504, 506, 508, and 510 show, respectively, and relative to FIGS. 5C and 5D, less scattered Lorenz plots, less scattered normalized Lorenz plots, less scatted RR interval histograms, and low episode duration.

FIG. 5G shows an example of combined image 550, which corresponds to true AF, and FIG. 5H shows an example of combined image 552, which corresponds to not true AF. As can be seen in combined image 550, the sub-image corresponding to normalized R-wave amplitudes does not show a single enhanced P-wave, and the sub-image corresponding to a Lorenz plot shows a large Lorenz plot scatter. The sub-image corresponding to a normalized Lorenz plot shows a high AF burden History (Hx), and the sub-image-corresponding to a multi-scale RR histogram shows a broad RR histogram at multiple scales. Finally, the sub-image corresponding to an episode duration indicates a long episode duration. In contrast, as can be seen in combined image 552, the sub-image corresponding to normalized R-wave amplitudes shows an enhanced P-wave, and the sub-image corresponding to a Lorenz plot shows a low Lorenz plot scatter. The sub-image corresponding to a normalized Lorenz plot shows no high AF burden History, and the sub-image-corresponding to a multi-scale RR histogram shows a not broad RR histogram at multiple scales. Finally, the sub-image corresponding to an episode duration shows a short episode duration.

Monitoring system 450 may apply the generated images, like those described with respect to FIGS. 5A-5F, as inputs to a selected one or more machine learning models. In the example illustrated by FIG. 4, monitoring system 450 may apply episode data to one or more arrhythmia classification machine learning models 452 (also described herein as arrhythmia classification models 452). Arrhythmia classification models 452 may include, as examples, neural networks, such as deep neural networks, which may include convolutional neural networks, multi-layer perceptrons, and/or echo state networks, as examples. The various characteristics of sub-images discussed above with respect to FIGS. 5C-5F represent just a few of the many characteristics that arrythmia classification models 452 may be trained to identify to determine if an arrythmia type, such as AF, occurred during an episode.

Arrhythmia classification machine learning models 452 may be configured to output, for each of a plurality of arrhythmia type classifications, values indicative of the likelihood that an arrhythmia of the type occurred at any point during the episode. Monitoring system 450 may apply configurable thresholds (e.g., 50%, 75%, 90%, 95%, 99%) to the likelihood values to identify the episode as including one or more arrhythmia types, e.g., based on the likelihood for that classification meeting or exceeding the threshold.

In some examples, arrhythmia classification machine learning models 452 are trained with training data, e.g., training images, generated from cardiac EGM or other episode data for a plurality of patients, labeled with descriptive metadata. The training images may be formatted similarly to image template 500 of FIG. 5A or in some other manner. For example, during a training phase, monitoring system 450 processes a plurality of training images. Typically, the plurality of training images are derived from cardiac EGM waveforms from a plurality of different patients, but may be from a single patient. Each image is labeled with one or more episodes of arrhythmia of one or more types.

For example, a training image may include a plurality of segments, each segment labeled with a descriptor that specifies an absence of arrhythmia or a presence of an arrhythmia of a particular classification (e.g., bradycardia, pause, tachycardia, atrial fibrillation, atrial flutter, atrioventricular block, or ventricular fibrillation). In some examples, a clinician labels the presence of arrhythmia in each image by hand. In some examples, the presence of arrhythmia in each image is labeled according to classification by a cardiac EGM feature delineation algorithm, e.g., similar to the techniques used by IMD 10 to identify arrhythmias based on rates, intervals, and morphological features derived from the cardiac EGM.

Monitoring system 450 may operate to convert the training data, e.g., the training images, into vectors and multi-dimensional arrays upon which monitoring system 450 may apply mathematical operations, such as linear algebraic, nonlinear, or alternative computation operations. Monitoring system 450 uses the training data to teach the one or more arrhythmia classification machine learning models 452 to weigh different features depicted in the images. In some examples, monitoring system 450 uses the training images to teach the machine learning model to apply different coefficients that represent one or more features in a cardiac EGM as having more or less importance with respect to an occurrence of a cardiac arrhythmia of a particular classification. By processing numerous such images labeled with episodes of arrhythmia, monitoring system 450 may build and train one or more arrhythmia classification machine learning models 452 to receive images depicting features of cardiac EGM data from a patient, such as patient 4 of FIG. 1, that monitoring system 450 has not previously analyzed, and process such image to detect the presence or absence of arrhythmia types of different classifications in the patient with a high degree of accuracy. Typically, the greater the amount of cardiac EGM data on which the one or more arrhythmia classification machine learning models 452 is trained, the higher the accuracy of the machine learning models in detecting or classifying cardiac arrhythmia in new cardiac EGM data.

After monitoring system 450 has trained one or more arrhythmia classification machine learning models 452, monitoring system 450 may receive episode data, such as cardiac electrogram EGM data, for a particular patient, such as patient 4. Monitoring system 450 generates an image based on the episode data and applies the one or more trained arrhythmia classification machine learning models 452 to the generated image to determine whether one or more arrhythmia types occurred at any point during the episode. As explained above, the image may include image data that depicts one or more features of the cardiac EGM data instead of, or in addition to, the raw cardiac EGM data itself. The one or more features may be obtained via feature delineation performed by IMD 10 and/or monitoring system 450. The features may include, e.g., one or more of heart rates, inter-depolarization intervals, other intervals between features of the cardiac EGM, one or more amplitudes, widths or morphological features of the QRS wave or other features of the cardiac EGM, variability of any of these features, T-wave alternans, or other types of cardiac features not expressly described herein. In such example implementations, monitoring system 450 may train the one or more arrhythmia classification machine learning models 452 via training images that include depictions of a plurality of training cardiac features labeled with episodes of arrhythmia, instead of or in addition to the plurality of cardiac EGM waveforms labeled with episodes of arrhythmia as described above.

In further examples, monitoring system 450 may generate, from the cardiac EGM data, an intermediate representation of the cardiac EGM data and include a depiction of this intermediate representation in the depicted image. For example, monitoring system 450 may apply one or more of signal processing, downsampling, normalization, signal decomposition, wavelet decomposition, filtering, noise reduction, or neural-network based feature representation operations to the cardiac electrogram data to generate the intermediate representation of the cardiac electrogram data. Monitoring system 450 may process images of such intermediate representation of the cardiac EGM data to detect and classify arrhythmias of various types in patient 4. Furthermore, monitoring system 450 may train the one or more arrhythmia classification machine learning models 452 via a plurality of training intermediate representations labeled with episodes of arrhythmia, instead of the plurality of raw cardiac EGM waveforms labeled with episodes of arrhythmia as described above. The use of such intermediate representations of the cardiac EGM data may allow for the training and development of lighter-weight, less computationally complex arrhythmia classification machine learning models 452 by monitoring system 450. Further, the use of such intermediate representations of the cardiac electrogram data may require smaller network is fewer parameters or less iterations and fewer training data to build an accurate machine learning model, as opposed to the use of raw cardiac EGM data to train the machine learning model.

In some examples, based on the application of images depicting episode data to the one or more arrhythmia classification machine learning models 452, monitoring system 450 may derive, for each of the arrhythmia type classifications, class activation data indicating varying likelihoods of the classification over the period of time of the episode's waveform. For a given arrhythmia type, the amplitude of such likelihood values at different times corresponds to the probability that an arrhythmia is occurring at that time, with higher values corresponding to higher probability. In some instances, monitoring system 450 may generate a combined image using 120 seconds worth of data and use arrythmia classification models 452 to determine if an AF episode occurred during those 120 seconds. In some implementations, monitoring system 450 may be configured to further determine a smaller time window within the 120 seconds during which the AF was detected by, for example, generating additional images using smaller time intervals to determine which time interval caused the AF classification. In many use cases, however, such additional classification may be unnecessary.

Class activation mapping may make it possible to identify regions of an input time series, e.g., of cardiac EGM data, that constitute the reason for the time series being given a particular classification by the one or more arrhythmia classification machine learning models 452. A class activation map for a given classification may be a univariate time series where each element (e.g., at each timestamp at the sampling frequency of the input time series) may be a weighted sum or other value derived from the outputs of an intermediate layer of a neural network or other machine learning model. The intermediate layer may be a global average pooling layer and/or last layer prior to the output layer neurons for each classification.

Monitoring system 450 may display, e.g., via output device(s) 412 and/or communication with another device via communication circuitry 406, a graph of the activation data over the time period of the episode. In some examples, monitoring system 450 may display the class activation data in conjunction with, e.g., on the same screen and at the same time, as the input cardiac EGM. While the one or more arrhythmia classification machine learning models 452 may be configured to provide an output that indicates a likelihood of different arrhythmia type classifications occurring during the episode as a whole, the class activation data may allow monitoring system 450 and/or a user to identify a time during an episode and point during the cardiac EGM at which one or more arrhythmias of one or more types likely occurred.

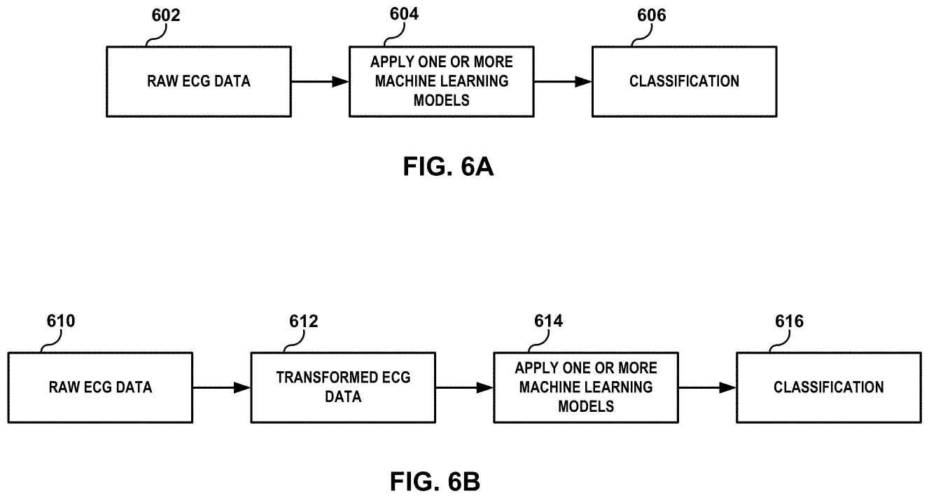

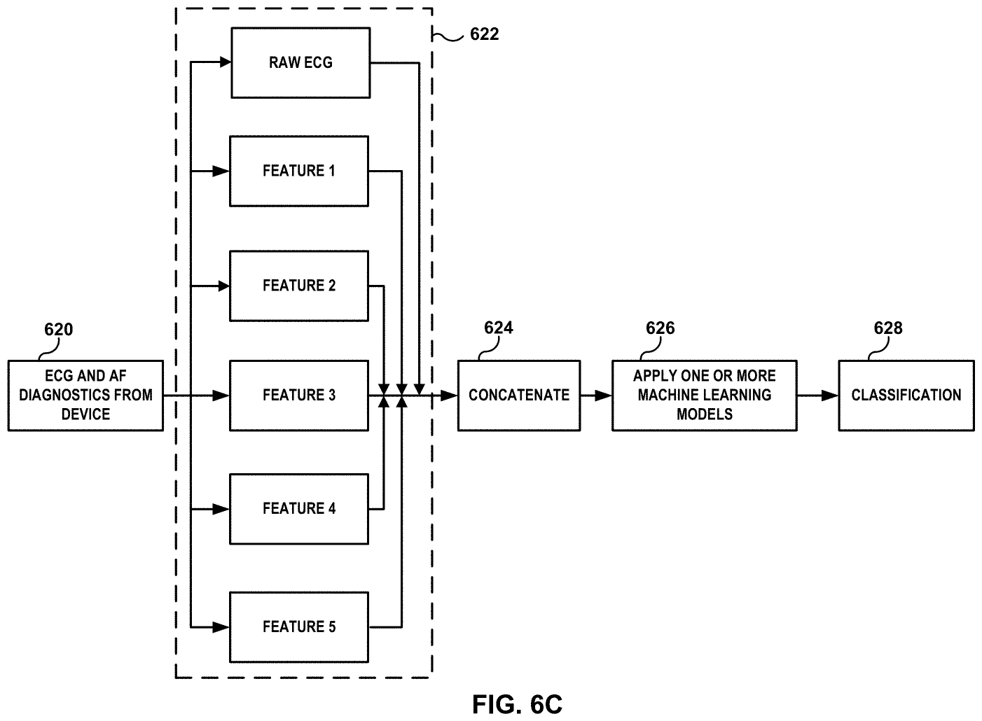

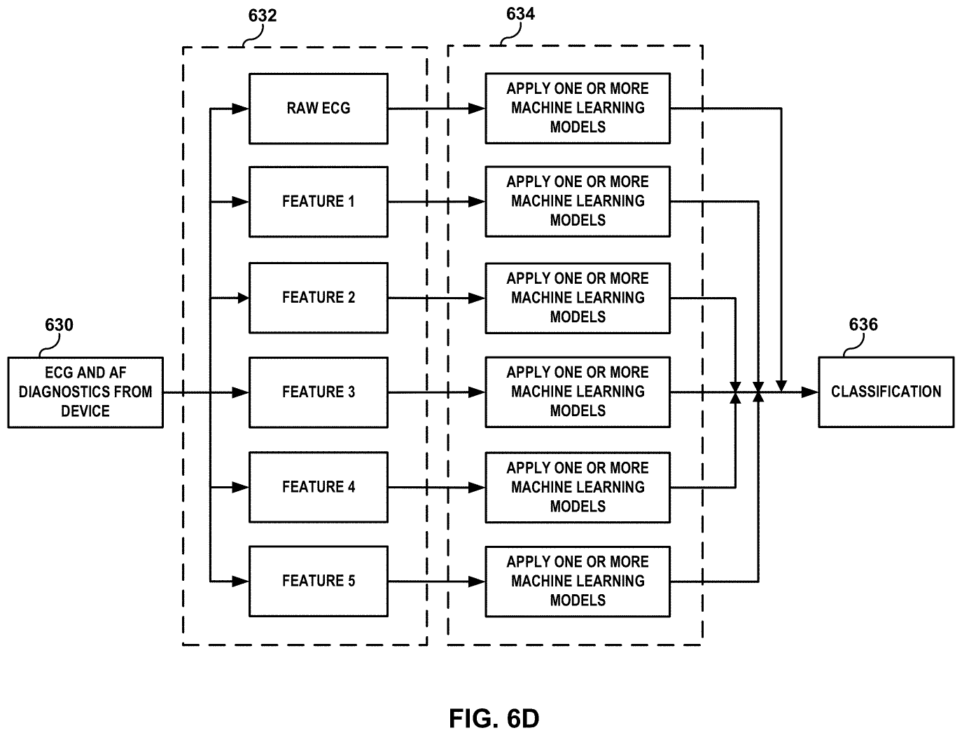

FIGS. 6A-6D are process flow schematics illustrating an example operation for utilizing machine learning models to detect cardiac arrhythmias in accordance with the techniques of the disclosure. In the example of FIG. 6A, monitoring system 450 receives raw ECG data for a cardiac episode (602), applies one or more machine learning models to the raw ECG data (604), and classifies the cardiac episode based on the determinations of the one or more machine learning models (606). In the example of FIG. 6B, monitoring system 450 receives raw ECG data for a cardiac episode (610), transforms the raw ECG data (612), applies one or more machine learning models to the transformed ECG data (614), and classifies the cardiac episode based on the determinations of the one or more machine learning models (616).

In the example of FIG. 6C, monitoring system 450 receives raw ECG data and diagnostic data for a cardiac episode (620), extracts features from the raw ECG data and diagnostic data (622), concatenates the extracted features (624), applies one or more machine learning models to the concatenated features (626), and classifies the cardiac episode based on the determinations of the one or more machine learning models (628). The extracted features may, for example, include any or all of a Lorenz plot, P-waves in long cycle, an atrial rate estimation, an enhanced p-wage morphology, a multi-scale RR histogram, an AF burden history, and an episode duration. Monitoring system 450 may concatenate the extracted features by generating a combined image like the images discussed with respect to FIGS. 5A-5F or in some other manner. As shown in the example of FIG. 6C, monitoring system 450 may also include the raw ECG data in the combined image.

In the example of FIG. 6D, monitoring system 450 receives raw ECG data and diagnostic data for a cardiac episode (630), extracts features from the raw ECG data and diagnostic data (632), applies one or more machine learning models individually to the extracted features (634), and classifies the cardiac episode based on the determinations of the one or more machine learning models (636). The extracted features may, for example, include any or all of a Lorenz plot, P-waves in long cycle, an atrial rate estimation, an enhanced p-wage morphology, a multi-scale RR histogram, an AF burden history, and an episode duration. As shown in the example of FIG. 6D, monitoring system 450 may classify the cardiac episode based on the raw ECG data. Monitoring system 450 may, for example, make a final classification for the cardiac episode data using a weighted voting mechanism or some other such process.

The processes of FIGS. 6A-6D are not mutually exclusive and may be used in conjunction with one another. As one example, monitoring system 450 may determine a first classification for cardiac episode data using the process of FIG. 6A and determine a second classification for the cardiac episode data using the process of any of FIGS. 6B-6D. Monitoring system 450 may then make a final classification based on the first classification and the second classification using a voting mechanism or some other such process.

The techniques of this disclosure described in FIGS. 6A-6D and elsewhere are not limited to any particular type of machine learning models. As part of applying one or more machine learning models, monitoring system 450 may, for example, perform convolution feature engineering, where convolution layers may automatically train weights for each of the filters to define what features works the best. In conventional machine learning, the features of image processing and computer vision, like edge detectors, orientation detectors, etc., are user defined. In deep learning, the convolution layers starts with random weights for individual convolutions filters, and then iterate to change those weights to reduce the loss function. At the end, the network has defined feature filters automatically. The techniques described herein may be implemented using either conventional machine learning or deep learning.

As part of applying one or more machine learning models, monitoring system 450 may, for example, also utilize a flattening layer to convert a 2D image into a 1D signal, thus losing the spatial characteristics. This may, for example be done prior to a dense and classification layer.

Other aspects of applying one or more machine learning models may include convolution, linear rectification, and max pooling. Convolution essentially refers to a applying an XbyY pixel 2D filter (or a 1D filter), with XY weights for each element in the filter. Monitoring system 450 may perform pixel by pixel multiplication over an area of an input image, obtain a sum of the filtered elements, and add a bias to obtain an activation map. The training process of the network updates those weights of the filter and the bias to reduce the loss function. Linear rectification utilizes a rectifier to keep the positive values and make all negative values be equal to zero. Monitoring system 450 may, for example, use a nonlinear function after convolution for better convergence. Max Pooling generally refers to a downsampling step where the max value over a spatial area is obtained. For example, to downsample by a factor of 2, the max value of a 2×2 area of the image is obtained and that is the one pixel that represents that area. Thus a 64×64 image may be downsampled, using max pooling, to obtain a 32×32 image, filled with the max value of each 2×2 spatial unit.

As indicated above, the techniques of this disclosure are not limited to any particular machine learning models. The above described techniques merely represent examples of the processes that monitoring system 450 may perform when applying one or more machine learning models to implement the techniques of this disclosure.

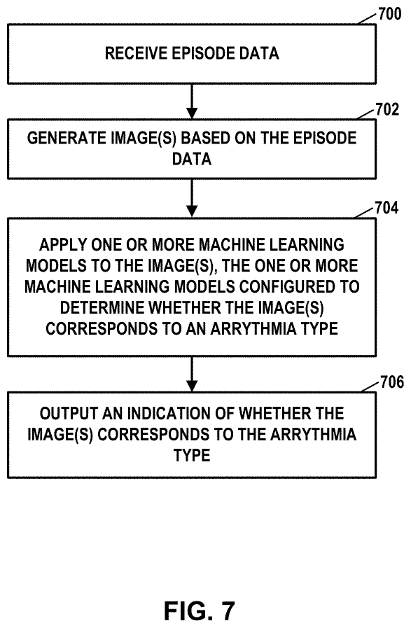

FIG. 7 is a flow diagram illustrating an example operation for analyzing ECG episode data to identify the presence of an arrythmia. According to the example illustrated by FIG. 7, monitoring system 450 receives episode data recorded by IMD 10 for an episode (700). The episode data includes, for example, a cardiac electrogram sensed by IMD 10 during a period of time. Monitoring system 450 generates one or more images based on the episode data (702). Each of the images may be associated with an interval within the period of time. The period of time may, for example, be 2 minutes or any other suitable period. In some examples, the interval and the period of time may be the same, meaning the generated image may be based on data for the entire period of the episode. In other examples, the interval may be less than the period of the episode data, meaning that the image generated based on the episode data may be based on data for the less than the entire period of the episode. Monitoring system 450 may, for example, generate a first image for a first interval of the period and a second image for a second interval of the period. For instance, if the period is 2 minutes, then monitoring system 450 may generate a first image corresponding to the first 1 minute interval of the 2 minute period and generate a second image corresponding to the second 1 minute interval of the 2 minute period.

In some examples, monitoring system 450 may generate multiple images that correspond to the same interval within a period or to overlapping intervals within a period. The multiple images may, for example, include different features or a different arrangement of sub-pictures within the image.

Monitoring system 450 applies one or more machine learning models to the one or more images, the one or more machine learning models configured to determine whether the one or more image corresponds to an arrythmia type (704). If using multiple images, monitoring system 450 may determine whether the one or more images correspond to an arrythmia type based on a majority vote of the image classification results, a super majority vote of the image classification results, a confidence-weighted vote of the image classification results, or some other such mechanism. Similarly, if using multiple machine learning models, monitoring system 450 may determine whether the one or more images correspond to an arrythmia type based on a majority vote of the machine learning models, a super majority vote of the machine learning models, a confidence-weighted vote of the machine learning models, or some other such mechanism. If using multiple machine learning models, not all of the machine learning models necessarily need to be based on images of features extracted from the raw ECG data. Monitoring system 450 could, for example, apply one or machine learning models to images of features extracted from the raw ECG data in addition to one or more machine learning models to the raw ECG data.

Monitoring system 450 outputs an indication of whether the image corresponds to the arrythmia type (706). The indication output by monitoring system 450 may take any one of numerous different forms. For example, monitoring system 450 may output a likelihood value or probability value that represents the likelihood or probability that a certain arrhythmia type occurred at a point during the period of time of the episode. Such a value may be a percentage (e.g., 0-100%) value, a binary determination (e.g., “yes” an arrythmia occurred or “no” an arrythmia did not occur), a numeric representation (e.g., 1-10), or any type of arbitrary type of descriptor (e.g., low, moderate, high). In some example system, monitoring system 450 may only receive episode data that another system, such as IMD 10, has already classified as being representative of an arrythmia. In such an instance, the output may be an affirmation or negation of the determination made by the other system.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques and systems described herein.

Example 1. A computer-implemented method comprising: receiving, by processing circuitry of a medical device system, episode data for an episode stored by a medical device of a patient, wherein the episode data comprises a cardiac electrogram sensed by the medical device during a period of time; generating an image based on the episode data, wherein the image is associated with an interval within the period of time; applying, by the processing circuitry, one or more machine learning models to the image, the one or more machine learning models configured to determine whether the image corresponds to an arrythmia type; and outputting an indication of whether the image corresponds to the arrythmia type.

Example 2. The method of example 1, wherein generating the image based on the episode data comprises: identifying R-wave amplitudes in the cardiac electrogram; performing a log transformation on the R-wave amplitudes to determine normalized R-wave amplitudes; and including, in the image, an image of the normalized R-wave amplitudes.

Example 3. The method of example 1 or 2, wherein the image comprises an image of a Lorenz plot determined from the cardiac electrogram.

Example 4. The method of any of examples 1-3, wherein generating the image based on the episode data comprises: determining, from the cardiac electrogram, a first sub-image corresponding to a first feature of the cardiac electrogram; determining, from the cardiac electrogram, a second sub-image corresponding to a second feature of the cardiac electrogram; including the first sub-image in a first region of the image; and including the second sub-image in a second region of the image.

Example 5. The method of example 4, wherein the first sub-image corresponding to the first feature of the cardiac electrogram comprises an image of a Lorenz plot determined from the cardiac electrogram.

Example 6. The method of example 4, wherein the first sub-image corresponding to the first feature of the cardiac electrogram comprises an image of a normalized Lorenz plot determined from the cardiac electrogram.

Example 7. The method of example 4, wherein the first sub-image corresponding to the first feature of the cardiac electrogram comprises an image of a multi-scale RR histogram determined from the cardiac electrogram.

Example 8. The method of example 4, wherein the first sub-image corresponding to the first feature of the cardiac electrogram comprises an image of an episode duration encoding block determined from the cardiac electrogram.

Example 9. The method of any of examples 1-8: wherein the medical device of the patient assigned an arrythmia type classification to the episode, the arrythmia type classification indicating that the episode corresponds to the arrythmia type; and wherein outputting the indication of whether the image corresponds to the arrythmia type comprises outputting an indication of whether the arrythmia type classification determined by the medical device of the patient is true or false.

Example 10. The method of any of examples 1-9, wherein the arrhythmia type comprises one of atrial fibrillation, atrial tachycardia, or atrial flutter.

Example 11. The method of any of examples 1-9, wherein the arrhythmia type comprises bradycardia, pause, ventricular tachycardia, ventricular fibrillation, supraventricular tachycardia, atrial flutter, sinus tachycardia, premature ventricular contraction, premature atrial contraction, wide complex tachycardia, and atrioventricular block.

Example 12. A medical system comprising: communication circuitry configured to receive episode data for an episode sensed by a medical device of a patient, wherein the episode data comprises a cardiac electrogram sensed by the medical device during a period of time; processing circuitry configured to: generate an image based on the episode data, wherein the image is associated with an interval within the period of time; apply, by the processing circuitry, one or more machine learning models to the image, the one or more machine learning models configured to determine whether the image corresponds to an arrythmia type; and output an indication of whether the image corresponds to the arrythmia type.

Example 13. The medical system of example 12, wherein to generate the image based on the episode data, the processing circuitry is configured to: identify R-wave amplitudes in the cardiac electrogram; perform a log transformation on the R-wave amplitudes to determine normalized R-wave amplitudes; and include, in the image, an image of the normalized R-wave amplitudes.

Example 14. The medical system of example 12 or 13, wherein the image comprises an image of a Lorenz plot determined from the cardiac electrogram.

Example 15. The medical system of any of examples 12-14, wherein to generate the image based on the episode data, the processing circuitry is configured to: determine, from the cardiac electrogram, a first sub-image corresponding to a first feature of the cardiac electrogram; determine, from the cardiac electrogram, a second sub-image corresponding to a second feature of the cardiac electrogram; include the first sub-image in a first region of the image; and include the second sub-image in a second region of the image.

Example 16. The medical system of example 15, wherein the first sub-image corresponding to the first feature of the cardiac electrogram comprises an image of a Lorenz plot determined from the cardiac electrogram.

Example 17. The medical system of example 15, wherein the first sub-image corresponding to the first feature of the cardiac electrogram comprises an image of a normalized Lorenz plot determined from the cardiac electrogram.

Example 18. The medical system of example 15, wherein the first sub-image corresponding to the first feature of the cardiac electrogram comprises an image of a multi-scale RR histogram determined from the cardiac electrogram.

Example 19. The medical system of example 15, wherein the first sub-image corresponding to the first feature of the cardiac electrogram comprises an image of an episode duration encoding block determined from the cardiac electrogram.

Example 20. The medical system of any of examples 12-19: wherein the medical device of the patient assigned an arrythmia type classification to the episode, the arrythmia type classification indicating that the episode corresponds to the arrythmia type; and wherein to output the indication of whether the image corresponds to the arrythmia type, the processing circuitry is configured to output an indication of whether the arrythmia type classification determined by the medical device of the patient is true or false.

Example 21. The medical system of any of examples 12-20, wherein the arrhythmia type comprises one of atrial fibrillation, atrial tachycardia, or atrial flutter.

Example 22. The medical system of any of examples 12-20, wherein the arrhythmia type comprises bradycardia, pause, ventricular tachycardia, ventricular fibrillation, supraventricular tachycardia, atrial flutter, sinus tachycardia, premature ventricular contraction, premature atrial contraction, wide complex tachycardia, and atrioventricular block.

Example 23. A computer-readable storage medium storing instructions that when executed by one or more processors cause the one or more processors to: receive episode data for an episode stored by a medical device of a patient, wherein the episode data comprises a cardiac electrogram sensed by the medical device during a period of time; generate an image based on the episode data, wherein the image is associated with an interval within the period of time; apply one or more machine learning models to the image, the one or more machine learning models configured to determine whether the image corresponds to an arrythmia type; and output an indication of whether the image corresponds to the arrythmia type.

Various examples have been described. These and other examples are within the scope of the following claims.

What is claimed is:

1. A computer-implemented method comprising:
receiving, by processing circuitry of a medical device system, episode data for an episode stored by a medical device of a patient, wherein the episode data comprises a cardiac electrogram sensed by the medical device during a period of time;
generating an image based on the episode data, wherein the image is associated with an interval within the period of time;
applying, by the processing circuitry, one or more machine learning models to the image, the one or more machine learning models configured to determine whether the image corresponds to an arrythmia type; and
outputting an indication of whether the image corresponds to the arrythmia type.

2. The method of claim 1, wherein generating the image based on the episode data comprises:
identifying R-wave amplitudes in the cardiac electrogram;
performing a log transformation on the R-wave amplitudes to determine normalized R-wave amplitudes; and
including, in the image, an image of the normalized R-wave amplitudes.

3. The method of claim 1, wherein the image comprises an image of a Lorenz plot determined from the cardiac electrogram.

4. The method of claim 1, wherein generating the image based on the episode data comprises:
determining, from the cardiac electrogram, a first sub-image corresponding to a first feature of the cardiac electrogram;
determining, from the cardiac electrogram, a second sub-image corresponding to a second feature of the cardiac electrogram;
including the first sub-image in a first region of the image; and
including the second sub-image in a second region of the image.

5. The method of claim 4, wherein the first sub-image corresponding to the first feature of the cardiac electrogram comprises an image of a Lorenz plot determined from the cardiac electrogram.

6. The method of claim 4, wherein the first sub-image corresponding to the first feature of the cardiac electrogram comprises an image of a normalized Lorenz plot determined from the cardiac electrogram.

7. The method of claim 4, wherein the first sub-image corresponding to the first feature of the cardiac electrogram comprises an image of a multi-scale RR histogram determined from the cardiac electrogram.

8. The method of claim 4, wherein the first sub-image corresponding to the first feature of the cardiac electrogram comprises an image of an episode duration encoding block determined from the cardiac electrogram.

9. The method of claim 1:
wherein the medical device of the patient assigned an arrythmia type classification to the episode, the arrythmia type classification indicating that the episode corresponds to the arrythmia type;
wherein outputting the indication of whether the image corresponds to the arrythmia type comprises outputting an indication of whether the arrythmia type classification determined by the medical device of the patient is true or false.

10. The method of claim 1, wherein the arrhythmia type comprises one of atrial fibrillation, atrial tachycardia, or atrial flutter.

11. The method of claim 1, wherein the arrhythmia type comprises bradycardia, pause, ventricular tachycardia, ventricular fibrillation, supraventricular tachycardia, atrial flutter, sinus tachycardia, premature ventricular contraction, premature atrial contraction, wide complex tachycardia, and atrioventricular block.

12. A medical system comprising:
communication circuitry configured to receive episode data for an episode sensed by a medical device of a patient, wherein the episode data comprises a cardiac electrogram sensed by the medical device during a period of time;
processing circuitry configured to:
generate an image based on the episode data, wherein the image is associated with an interval within the period of time;
apply, by the processing circuitry, one or more machine learning models to the image, the one or more machine learning models configured to determine whether the image corresponds to an arrythmia type; and
output an indication of whether the image corresponds to the arrythmia type.

13. The medical system of claim 12, wherein to generate the image based on the episode data, the processing circuitry is configured to:
identify R-wave amplitudes in the cardiac electrogram;
perform a log transformation on the R-wave amplitudes to determine normalized R-wave amplitudes; and
include, in the image, an image of the normalized R-wave amplitudes.

14. The medical system of claim 12, wherein the image comprises an image of a Lorenz plot determined from the cardiac electrogram.

15. The medical system of claim 12, wherein to generate the image based on the episode data, the processing circuitry is configured to:
determine, from the cardiac electrogram, a first sub-image corresponding to a first feature of the cardiac electrogram;
determine, from the cardiac electrogram, a second sub-image corresponding to a second feature of the cardiac electrogram;
include the first sub-image in a first region of the image; and
include the second sub-image in a second region of the image.

16. The medical system of claim 15, wherein the first sub-image corresponding to the first feature of the cardiac electrogram comprises an image of a Lorenz plot determined from the cardiac electrogram.

17. The medical system of claim 15, wherein the first sub-image corresponding to the first feature of the cardiac electrogram comprises an image of a normalized Lorenz plot determined from the cardiac electrogram.

18. The medical system of claim 15, wherein the first sub-image corresponding to the first feature of the cardiac electrogram comprises an image of a multi-scale RR histogram determined from the cardiac electrogram.

19. The medical system of claim 15, wherein the first sub-image corresponding to the first feature of the cardiac electrogram comprises an image of an episode duration encoding block determined from the cardiac electrogram.

20. The medical system of claim 12:
wherein the medical device of the patient assigned an arrythmia type classification to the episode, the arrythmia type classification indicating that the episode corresponds to the arrythmia type;
wherein to output the indication of whether the image corresponds to the arrythmia type, the processing circuitry is configured to output an indication of whether the arrythmia type classification determined by the medical device of the patient is true or false.

21. The medical system of claim 12, wherein the arrhythmia type comprises one of atrial fibrillation, atrial tachycardia, or atrial flutter.

22. The medical system of claim 12, wherein the arrhythmia type comprises bradycardia, pause, ventricular tachycardia, ventricular fibrillation, supraventricular tachycardia, atrial flutter, sinus tachycardia, premature ventricular contraction, premature atrial contraction, wide complex tachycardia, and atrioventricular block.

23. A computer-readable storage medium storing instructions that when executed by one or more processors cause the one or more processors to:
receive episode data for an episode stored by a medical device of a patient, wherein the episode data comprises a cardiac electrogram sensed by the medical device during a period of time;
generate an image based on the episode data, wherein the image is associated with an interval within the period of time;
apply one or more machine learning models to the image, the one or more machine learning models configured to determine whether the image corresponds to an arrythmia type; and
output an indication of whether the image corresponds to the arrythmia type.
